(19) United States
(12) Patent Application Publication
(10) Pub. No.: US 2025/0032757 A1
(43) Pub. Date: Jan. 30, 2025

(54) CATHETER SYSTEM AND RELATED DEVICES FOR INSTRUMENT DELIVERY

(71) Applicant: Becton, Dickinson and Company, Franklin Lakes, NJ (US)

(72) Inventors: Adam J. Boud, Bluffdale, UT (US); Weston F. Harding, Lehi, UT (US); Curtis H. Blanchard, Riverton, UT (US); Sridhaar Nandakumar, Tamil Nadu (IN); Prasad Govindaraj, Tamil Nadu (IN); Meenakshi Sankar Gomathinayagam, Tamil Nadu (IN); Jonathan Karl Burkholz, Salt Lake City, UT (US); S. Ray Isaacson, Layton, UT (US); Austin Newton, Layton, UT (US)

(21) Appl. No.: 18/227,725

(22) Filed: Jul. 28, 2023

(51) Int. Cl. A61M 25/06; A61M 39/10; A61M 39/22

(52) U.S. Cl. CPC A61M 25/0606; A61M 39/10; A61M 39/22

(57) ABSTRACT

An instrument delivery device to insert an instrument through a catheter assembly may include a housing, a connector coupled to the housing, a slider configured to move along a slot of the housing, and an instrument disposed within the housing and coupled to the slider. The instrument may be configured to advance beyond the connector in response to movement of the slider along the slot from a proximal position to a distal position. The connector may include a curved portion configured to change a direction of the instrument when the instrument is advanced through the connector. A catheter system may include a catheter adapter, which may include a side port. An annular valve may be disposed within the lumen and configured to seal the side port. The catheter system may include an instrument advancement feature configured to direct the instrument in a distal direction within the lumen of the catheter adapter.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of a catheter assembly including the catheter. Once placement of the needle has been confirmed, the clinician may temporarily occlude flow in the vasculature and remove the needle, leaving the catheter in place for future blood withdrawal or fluid infusion.

Blood withdrawal using a catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and positioning of a tip of the catheter to the vasculature. Due to this, catheters may often be used for acquiring a blood sample at a time of catheter placement but are much less frequently used for acquiring a blood sample during the catheter indwell period Therefore, when a blood sample is required, an additional needle stick is needed to provide vein access for blood collection, which may be painful for the patient and result in higher material costs. Accordingly, there is a need for catheter systems, devices, and methods that facilitate placement of instruments, such as, for example, flexible flow tubing for blood collection or a probe, in the vasculature of the patient without additional needle sticks.

The subject matter claimed herein is not limited to embodiments that solve any disadvantages or that operate only in environments such as those described above. Rather, this background is only provided to illustrate one example technology area where some implementations described herein may be practiced.

### SUMMARY

In some embodiments, the present disclosure generally relates to an instrument delivery device and/or a catheter system to facilitate instrument delivery into vasculature of a patient. In some embodiments, the present disclosure generally relates to a connector to facilitate instrument delivery into vasculature of a patient. In some embodiments, the present disclosure generally relates to a method to facilitate instrument delivery into vasculature of a patient.

In some embodiments, an instrument delivery device to insert an instrument through a catheter assembly may include a housing. In some embodiments, the housing may include a proximal end, a distal end, and a slot disposed between the proximal end of the housing and the distal end of the housing. In some embodiments, the instrument delivery device may include a connector coupled to the distal end of the housing. In some embodiments, the instrument delivery device may include a slider configured to move along the slot. In some embodiments, the instrument delivery device may include an instrument disposed within the housing and coupled to the slider.

In some embodiments, the instrument may be configured to advance beyond the connector in response to movement of the slider along the slot from a proximal position to a distal position. In some embodiments, the connector may include a curved portion configured to change a direction of the instrument when the instrument is advanced through the connector. In some embodiments, the instrument may include a flexible flow tube configured to extend through an indwelling catheter for blood collection. In some embodiments, the curved portion may be configured to change a direction of the instrument 90° when the instrument is advanced through the connector. In some embodiments, the curved portion may include a trough. In some embodiments, the curved portion may include an enclosed pathway.

In some embodiments, the enclosed pathway may be disposed within a straight end of the connector. In some embodiments, the connector may include a male luer configured to form a seal within the catheter assembly, and the straight end may extend from the male luer. In some embodiments, the enclosed pathway may be disposed within a curved end of the connector. In some embodiments, the connector may include the male luer configured to form the seal within the catheter assembly, and the curved end may extend from the male luer.

In some embodiments, the instrument delivery device may include a directional indicator to indicate to a clinician an orientation of the instrument delivery device with respect to the catheter assembly such that the curved portion is configured to direct the instrument in a distal direction within the catheter assembly. In some embodiments, the distal connector may include an insertion portion and multiple arms. In some embodiments, the insertion portion may be configured to insert into the catheter assembly. In some embodiments, multiple arms may be configured to snap onto an exterior of the catheter assembly.

In some embodiments, a catheter system may include a catheter adapter, which may include a distal end, a proximal end, a lumen extending through the distal end of the catheter adapter and the proximal end of the catheter adapter, and a side port disposed between the distal end and the proximal end. In some embodiments, the side port may be disposed at 90° with respect to a longitudinal axis of the catheter adapter. In some embodiments, the catheter system may include a catheter extending from the distal end of the catheter adapter.

In some embodiments, the catheter system may include an annular valve disposed within the lumen and configured to seal the side port. In some embodiments, the catheter system may include an instrument advancement feature. In some embodiments, when an instrument is inserted through the side port, the instrument advancement feature may be configured to direct the instrument in a distal direction within the lumen of the catheter adapter.

In some embodiments, the instrument advancement feature may include a ramp disposed within an interior of the annular valve. In some embodiments, the instrument advancement feature may include an attachment sleeve or adhesive that attaches a proximal end of the annular valve to an inner surface of the catheter adapter. In some embodiments, the instrument advancement feature may include a ramp on an inner surface of the catheter adapter. In some embodiments, the catheter system may include a needleless access connector, which may include a first end having a female luer. In some embodiments, the needleless access connector may include a second end coupled to the side port. In some embodiments, the second end of the connector may include the instrument advancement feature. In some embodiments, the instrument advancement feature may include the curved portion, and the curved portion may include a trough or an enclosed pathway.

In some embodiments, a catheter system may include a catheter adapter, which may include a distal end, a proximal end, a lumen extending through the distal end of the catheter adapter and the proximal end of the catheter adapter, and a side port disposed between the distal end and the proximal end. In some embodiments, the side port may be configured to be angled at less than 90° with respect to a longitudinal axis of the catheter adapter. In some embodiments, the catheter system may include a catheter extending from the distal end of the catheter adapter. In some embodiments, the catheter system may include an annular valve disposed within the lumen and configured to seal the side port. In some embodiments, the side port may be fixed with respect to the distal end of the catheter adapter and the proximal end of the catheter adapter at an angle less than 90° with respect to the longitudinal axis of the catheter adapter.

In some embodiments, the catheter system may include a cap coupled to the side port. In some embodiments, a central axis of the cap may be perpendicular to the longitudinal axis of the catheter adapter. In some embodiments, an inner surface of the cap may include a protrusion sealing the side port. In some embodiments, the side port may include a movable joint. In some embodiments, the side port may include a side port lumen having an asymmetric shape. In some embodiments, the side port may be fixed with respect to the distal end of the catheter adapter and the proximal end of the catheter adapter at an angle less than 90° with respect to the longitudinal axis of the catheter adapter. In some embodiments, the side port may extend from a top of catheter adapter. In some embodiments, the catheter system may include a septum disposed within the side port.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of a prior art blood draw device;

FIG. 1B is an upper perspective view of an end of the prior art blood draw device;

FIG. 1C is a cross-sectional view of the prior art blood draw device, in some instances;

FIG. 1D is a cross-sectional view of a prior art catheter system;

FIG. 2A is a cross-sectional view of an example catheter system, according to some embodiments;

FIG. 2B is a cross-sectional view of the catheter system coupled to an example instrument delivery device with an example instrument in and advanced position, according to some embodiments;

FIG. 2C is an upper perspective view of an example needleless access connector having an example trough, according to some embodiments;

FIG. 2D is a cross-sectional view of the catheter system including a needleless access connector having an enclosed pathway, according to some embodiments;

FIG. 2E is an upper perspective view of the needleless connector having the enclosed pathway, according to some embodiments;

FIG. 3A is a cross-sectional view of the catheter system including an example connector having an enclosed pathway, according to some embodiments;

FIG. 3B is an upper perspective view of the connector having the enclosed pathway, according to some embodiments;

FIG. 3C is an upper perspective view of the connector having the enclosed pathway, according to some embodiments;

FIG. 3D is a cross-sectional view of the catheter system illustrating an example connector having an enclosed pathway in a straight end, according to some embodiments;

FIG. 4A is an upper perspective view of the catheter system illustrating an example annular valve having a ramp therein, according to some embodiments;

FIG. 4B is a distal end view of the annular valve having the ramp therein, according to some embodiments;

FIG. 4C is a cross-sectional view of the catheter system illustrating the annular valve having a slit, according to some embodiments;

FIG. 4D is a cross-sectional view of the catheter system illustrating an example attachment sleeve securing a proximal end of the annular valve, according to some embodiments;

FIG. 4E is a cross-sectional view of the catheter system illustrating an example ramp of an example catheter adapter, according to some embodiments;

FIG. 4F is a cross-sectional view of the catheter system illustrating example opposing ramps of an example catheter adapter, according to some embodiments;

FIG. 5A is an upper perspective view of the catheter system, illustrating a side port fixed at an angle with respect to a longitudinal axis of the catheter system, according to some embodiments;

FIG. 5B is a cross-sectional view of the catheter system, illustrating the side port fixed at the angle, according to some embodiments;

FIG. 5C is a cross-sectional view of the catheter system, illustrating an example cap having an example protrusion, according to some embodiments;

FIG. 5D is a cross-sectional view of the catheter system, illustrating the cap having an inclined upper surface, according to some embodiments;

FIG. 5E is a top view of an example cap, illustrating an example oval shape, according to some embodiments;

FIG. 5F is a top view of an example cap, illustrating an example square or rectangular shape, according to some embodiments;

FIG. 6 is a cross-sectional view of the catheter system, illustrating an example aperture in a wall of the catheter adapter, according to some embodiments;

FIG. 7A is a cross-sectional view of the catheter system illustrating an example side port having a movable joint in a first position, according to some embodiments;

FIG. 7B is a cross-sectional view of the catheter system illustrating the side port having the movable joint in a second position, according to some embodiments;

FIG. 8A is an upper perspective view of the catheter adapter having a side port lumen that is asymmetric, according to some embodiments;

FIG. 8B is an upper perspective view of the catheter adapter having another side port lumen that is asymmetric, according to some embodiments;

FIG. 8C is an upper perspective view of the connector having an example directional indicator, according to some embodiments;

FIG. 8D is an upper perspective view of the catheter adapter, illustrating an example directional indicator, according to some embodiments;

FIG. 8E is an upper perspective view of the needleless access connector having an example directional indicator, according to some embodiments;

FIG. 9A is a cross-sectional view of the catheter system, illustrating an example septum actuator, according to some embodiments;

FIG. 9B is an enlarged cross-sectional view of a portion of the catheter system, illustrating the septum actuator and illustrating an example cap removed, according to some embodiments;

FIG. 9C is a cross-sectional view of the septum actuator, according to some embodiments;

FIG. 9D is a cross-sectional view of the needleless access connector coupled to the septum actuator, according to some embodiments;

FIG. 9E is a cross-sectional view of the connector coupled to the septum actuator, according to some embodiments;

FIG. 9F is a cross-sectional view of the catheter system coupled to the connector, according to some embodiments;

FIG. 9G is a cross-sectional view of the catheter adapter, illustrating an example septum, according to some embodiments;

FIG. 9H is a cross-sectional view of the catheter system, illustrating another example septum, according to some embodiments;

FIG. 10A is an upper perspective view of a prior art connector of a prior art blood draw device, according to some embodiments;

FIG. 10B is an upper perspective view of the catheter system illustrating the connector having multiple arms coupled to the cap, according to some embodiments;

FIG. 10C is a bottom schematic view of the connector having multiple arms, according to some embodiments; and

FIG. 10D is an upper perspective view of the catheter system illustrating the connector having multiple arms coupled to the catheter adapter, according to some embodiments.

### DESCRIPTION OF EMBODIMENTS

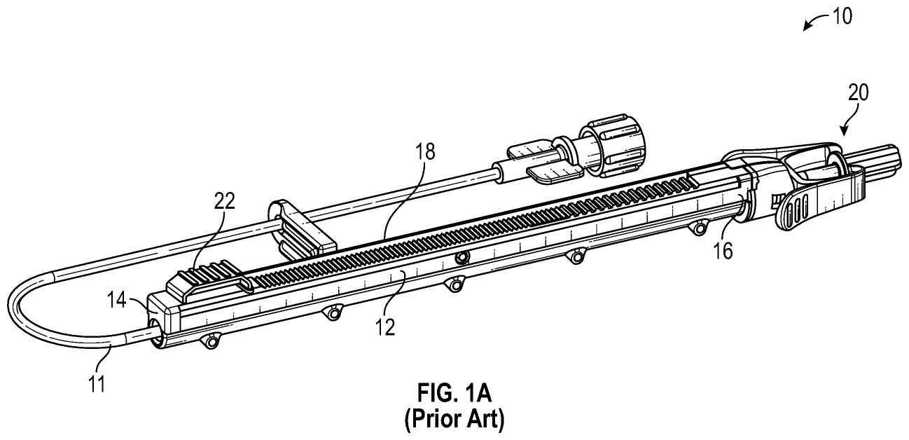

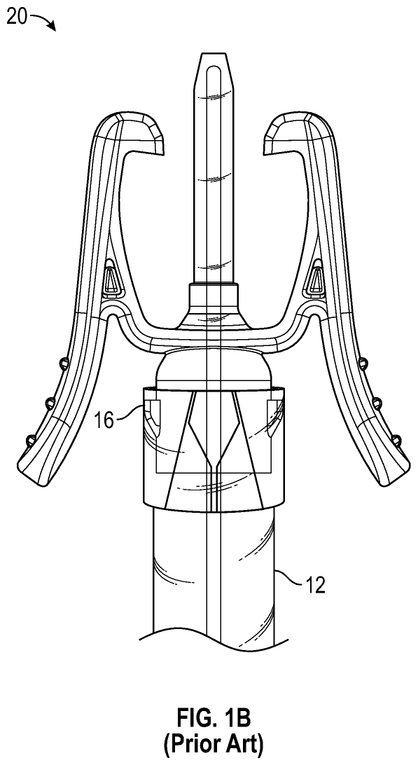

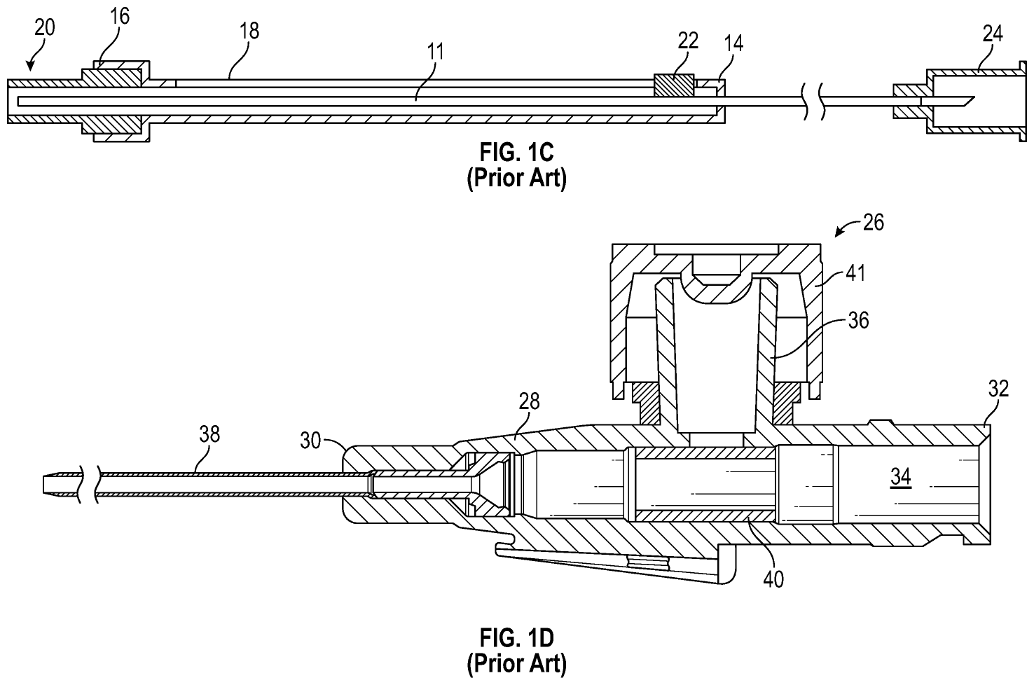

Referring now to FIGS. 1A-1C, a prior art blood draw device 10 is illustrated. The prior art blood draw device 10 may correspond to the PIVO™ Needle-Free Blood Draw Device available from Becton, Dickinson and Company of Franklin Lakes, New Jersey. The prior art blood draw device 10 advances a flexible flow tube 11 through an indwelling peripheral intravenous catheter and into a blood vessel to collect a blood sample. The prior art blood draw device 10 includes a housing 12, which includes a proximal end 14, a distal end 16, and a slot 18 disposed between the proximal end 14 of the housing 12 and the distal end 16 of the housing 12.

The prior art blood draw device 10 includes a connector 20 coupled to the distal end 16 of the housing 12. The prior art blood draw device 10 includes a slider 22 configured to move along the slot 18. The prior art blood draw device 10 includes the flexible flow tube 11 disposed within the housing 12 and coupled to the slider 22. The flexible flow tube 11 may advance through the indwelling peripheral intravenous catheter in response to movement of the slider 22 along the slot 18 from a proximal position to a distal position. When the flexible flow tube 11 is advanced through the indwelling peripheral intravenous catheter and is positioned within the blood vessel, blood may be collected through the flexible flow tube 11. In some instances, the flexible flow tube 11 may be advanced beyond a thrombus, valve, or other obstruction, extending to a new blood source. Thus, the flexible flow tube 11 may allow blood collection through an indwelling peripheral intravenous catheter, prolonging use of the indwelling peripheral intravenous catheter without further needle stick. The blood may be collected via an adapter 24 at a proximal end of the flexible flow tube 11. The adapter 24 may correspond to a BD VACUTAINER R LUER-LOK™ access device available from Becton, Dickinson and Company of Franklin Lakes, New Jersey.

Referring now to FIG. 1D, a prior art catheter system 26 is illustrated. The prior art catheter system 26 may correspond to the BD VENFLON™ Pro Safety Needle Protected IV Cannula, available from Becton, Dickinson and Company of Franklin Lakes, New Jersey. Currently the prior art catheter system 26 is not configured for use with the prior art blood draw device 10, and thus blood may not be collected easily from the prior art catheter system 26. The prior art catheter system 26 includes a catheter adapter 28, which includes a distal end 30, a proximal end 32, a lumen 34 extending through the distal end 30 of the catheter adapter 28 and the proximal end 32 of the catheter adapter 28, and a side port 36 disposed between the distal end 30 of the catheter adapter 28 and the proximal end 32 of the catheter adapter 28. The side port 36 is disposed at 90° with respect to a longitudinal axis of the catheter adapter 28. The prior art catheter system 26 may include a peripheral intravenous catheter 38 extending from the distal end 30 of the catheter adapter 28. The prior art catheter system 26 may include an annular valve 40 disposed within the lumen 34 and configured to seal the side port 36. A cap 41 is placed over the side port 36. The annular valve 40 is cylindrical and can collapse in various directions.

Coupling of the prior art blood draw device 10 to the side port 36 of the prior art catheter system 26 and advancement of the flexible flow tube 11 through the side port 36 and the peripheral intravenous catheter 38 would be difficult due to presence of the annular valve 40. Moreover, it is difficult to change a direction of the flexible flow tube 11 inserted into the side port 36 to exit the peripheral intravenous catheter 38 when the flexible flow tube 11 is advanced through the side port 36.

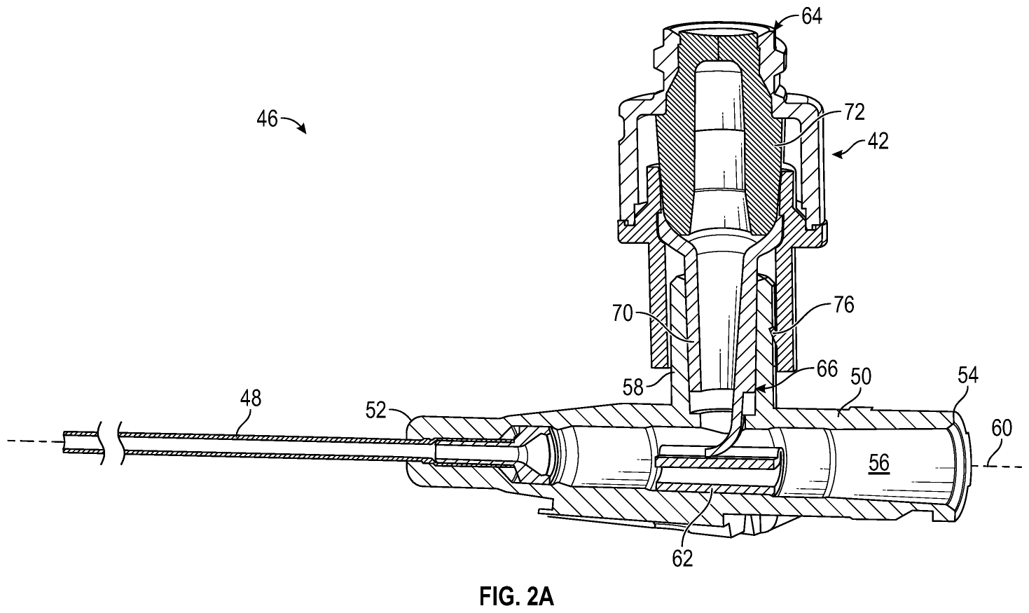

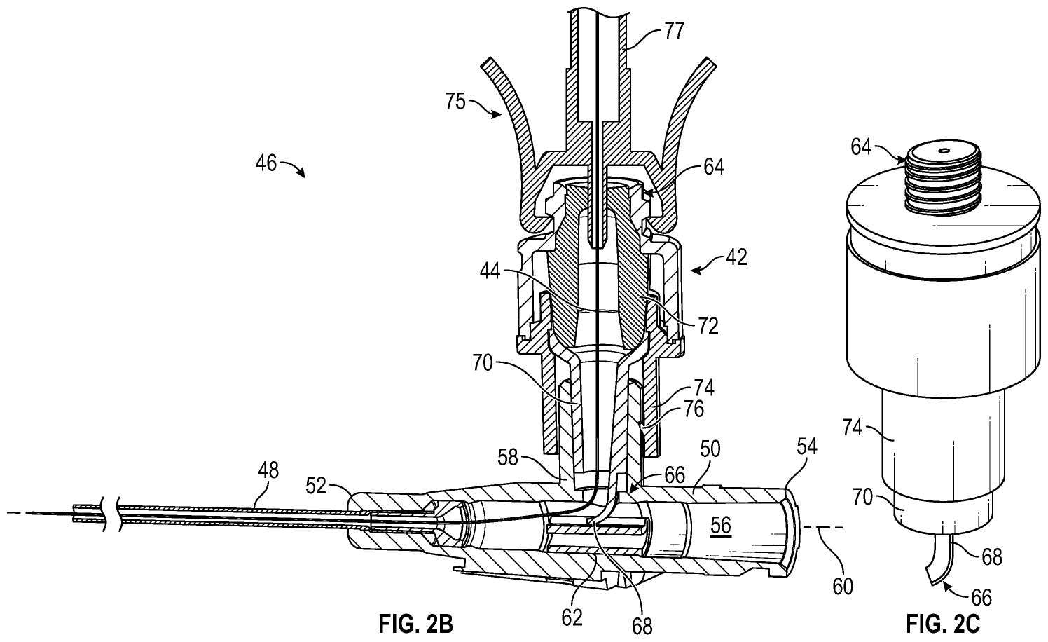

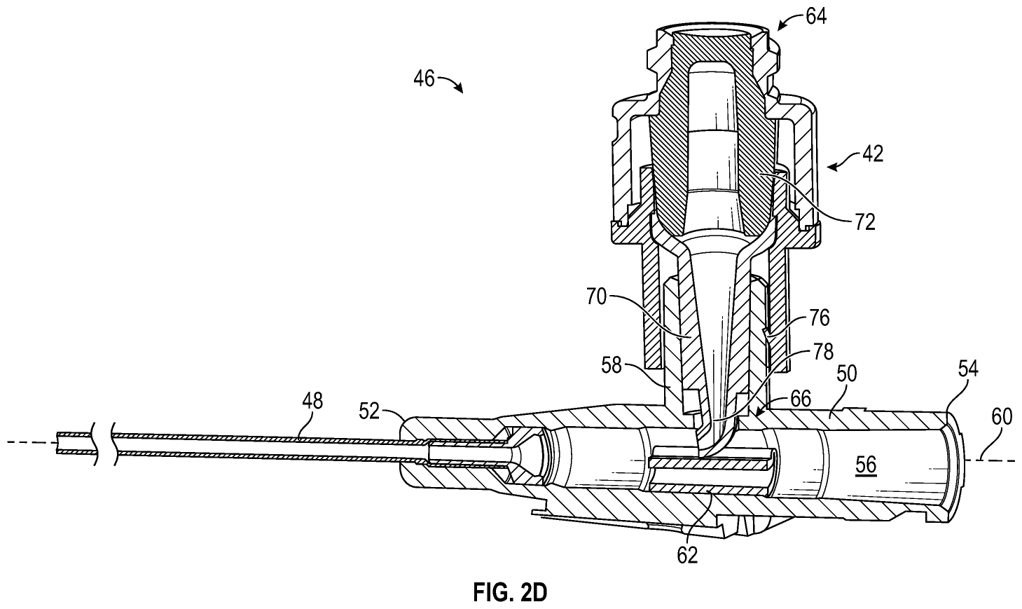

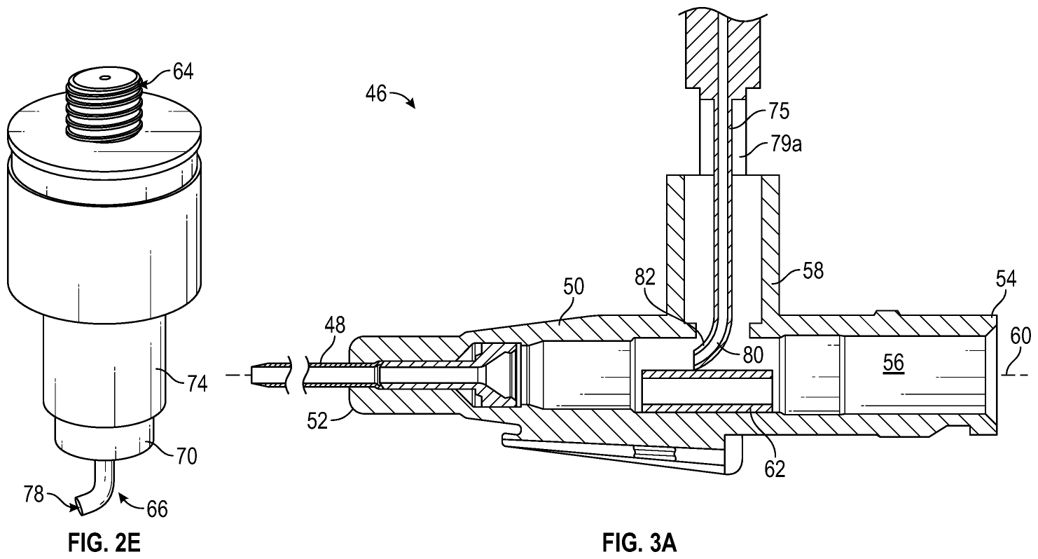

Referring now to FIGS. 2A-2E, in some embodiments, a needleless access connector 42 may facilitate advancement of an instrument 44 within a catheter system 46 that is ported. In some embodiments, the needleless access connector 42 may facilitate a directional change of the instrument 44 when the instrument 44 is advanced through the needleless access connector 42 such that the instrument 44 may be delivered distally through a catheter 48. In some embodiments, the catheter 48 may include a peripheral intravenous catheter, an arterial catheter, a midline catheter, a peripherally-inserted central catheter, or another type of catheter.

In some embodiments, the catheter system 46 may include a catheter assembly, which may include a catheter adapter 50 and the catheter 48. In some embodiments, the catheter adapter 50 may include a distal end 52, a proximal end 54, a lumen 56 extending through the distal end 52 of the catheter adapter 50 and the proximal end 54 of the catheter adapter 50, and a side port 58 disposed between the distal end 52 and the proximal end 54. In some embodiments, the side port 58 may be disposed at 90° with respect to a longitudinal axis 60 of the catheter adapter 50. In some embodiments, the catheter 48 may extend from the distal end 52 of the catheter adapter 50.

In some embodiments, the catheter system 46 may be similar or identical to the prior art catheter system 26 of FIG. 1D in terms of one or more features and/or operation. In some embodiments, the catheter system 46 may include an annular valve 62 disposed within the lumen 56 and configured to seal the side port 58. In some embodiments, the catheter system 46 may include an instrument advancement feature. In some embodiments, when the instrument 44 is inserted through the side port 58, the instrument advancement feature may be configured to direct the instrument 44 in a distal direction within the lumen 56 of the catheter adapter 50. In some embodiments, the instrument advancement feature may gradually change a direction of the instrument 44 such that the instrument 44 is not damaged.

In some embodiments, the needleless access connector 42 may include a first end 64 having a female luer. In some embodiments, the needleless access connector 42 may include a second end 66 coupled to the side port 58. In some embodiments, the second end 66 of the needleless access connector 42 may include the instrument advancement feature. For example, the needleless access connector 42 may include a curved portion configured to change a direction of the instrument 44 when the instrument 44 is advanced through the needleless access connector 42. In some embodiments, the curved portion may be configured to change a direction of the instrument 44 90° or approximately 90° (or another desired angle) when the instrument is advanced through the needleless access connector 42.

In some embodiments, the instrument 44 may include a flexible flow tube, a guidewire, a probe, a probe including one or more sensors, or another suitable instrument for delivery into vasculature of a patient. In some embodiments, the instrument advancement feature may facilitate movement of the instrument 44 through the catheter 48 when the catheter 48 is indwelling within the vasculature for blood collection, infusion, removal of blood clots, sensing one or more blood parameters, or another function.

As illustrated in FIGS. 2A-2C, in some embodiments, the curved portion may include a trough 68. In some embodiments, the needleless access connector 42 may include a male luer 70 configured to form a seal within the side port 58, and the trough 68 may extend from the male luer 70. In some embodiments, an outer surface of the male luer 70 may be generally cylindrical or tapered inwardly towards the second end 66 to contact an inner surface of the side port 58 along a length of the male luer 70 and form the seal. In some embodiments, the trough 68 may not be annular. In some embodiments, the trough 68 may extend from a proximal side of the male luer 70 or be disposed on a proximal side of the needleless access connector 42. In some embodiments, in response to the needleless access connector 42 being coupled to the side port 58, the trough 68 may depress the annular valve 62, which may facilitate advancement of the instrument 44 distally through the catheter adapter 50 and the catheter 48 into the vasculature.

In some embodiments, the needleless access connector 42 may include a septum 72, which may be disposed within the first end 64. In some embodiments, one or more of the first end 64, the second end 66, and the male luer 70 may be monolithically formed as a single unit or may be separate pieces coupled together. In some embodiments, the needleless access connector 42 may include an annular collar 74 surrounding the male luer 70. In some embodiments, an inner surface of the annular collar 74 may include a barb 76 configured to insert into an aperture in an outer surface of the side port 58, which may permanently secure the needleless access connector 42 to the side port 58 to maintain sterility. In some embodiments, the inner surface of the annular collar 74 may be threaded to the outer surface of the side port 58 or engaged in a slip fit with the outer surface of the side port 58.

In some embodiments, a connector 75 of an instrument delivery device 77 may be coupled to the first end 64. In some embodiments, the instrument delivery device 77 may include or correspond to the prior art blood draw device 10 in terms of one or more features and/or operation. In some embodiments, the connector 75 may include or correspond to the connector 20 of the prior art blood draw device 10 or another connector of another instrument delivery device for delivery an instrument into vasculature of a patient.

As illustrated in FIGS. 2D-2E, in some embodiments, the curved portion may include an enclosed pathway 78. In some embodiments, the enclosed pathway 78 may be disposed within a curved end of the needleless access connector 42, which may correspond to the second end 66. In some embodiments, the curved end may include an annular curved outer surface. In some embodiments, the needleless access connector 42 may include the male luer 70 configured to form the seal within the side port 58, and the curved end may extend from the male luer 70. In some embodiments, a diameter of the enclosed pathway 78 may be less than a diameter of the male luer 70. Thus, in some embodiments, there may be a stepped surface between the curved portion and the male luer 70. In some embodiments, in response to the needleless access connector 42 being coupled to the side port 58, the enclosed pathway 78 may depress the annular valve 62, which may facilitate advancement of the instrument 44 distally through the catheter adapter 50 and the catheter 48 into the vasculature.

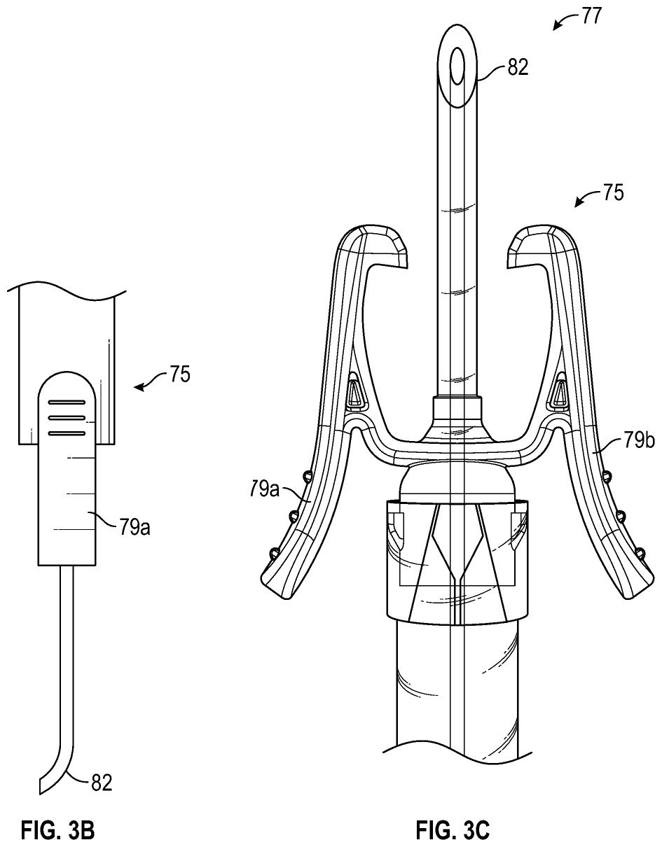

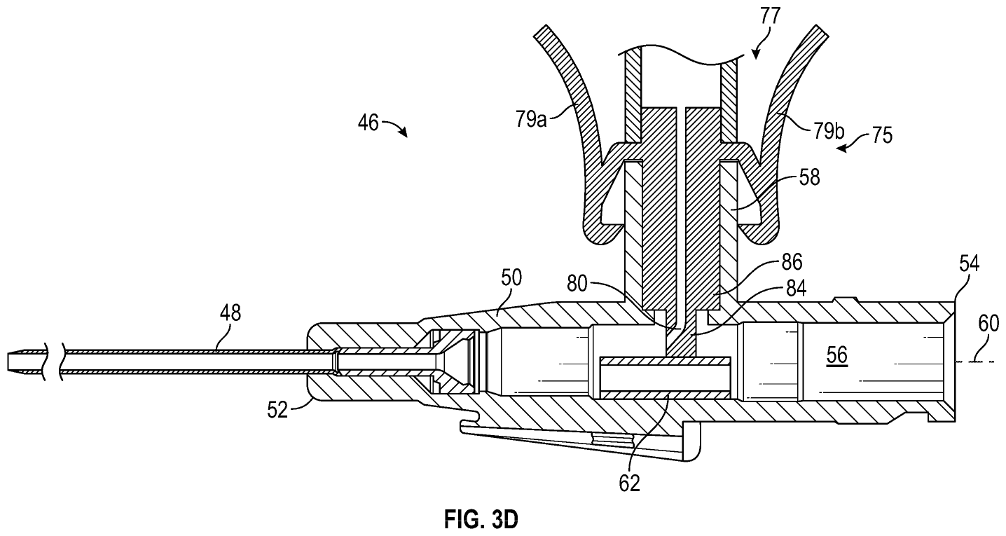

Referring now to FIGS. 3A-3D, in some embodiments, the connector 75 itself may facilitate advancement of the instrument 44 (see, for example, FIG. 2D) within the catheter system 46 that is ported. In some embodiments, the connector 75 may include or correspond to the connector 20 of the prior art blood draw device 10 or another connector of another instrument delivery device for delivery an instrument into vasculature of a patient. In some embodiments, the connector 75 may facilitate a directional change of the instrument 44 when the instrument 44 is advanced through the connector 75 such that the instrument 44 may be delivered distally through the catheter 48.

In some embodiments, the connector 75 may include opposing lever arms 79a, 79b. In some embodiments, distal ends of the opposing lever arms 79a, 79b may be configured to move apart from each other in response to pressure applied to proximal ends of the opposing lever arms 79a, 79b. In some embodiments, in response to removal of the pressure applied to the proximal ends of the opposing lever arms 79a, 79b, the distal ends may move closer to each other and clasp a portion of the catheter assembly, such as a needleless connector, another connector, or the side port 58, for example.

In some embodiments, the connector 75 may include a curved portion configured to change a direction of the instrument 44 when the instrument 44 is advanced through the connector 20. In some embodiments, the curved portion may correspond to the instrument advancement feature. In some embodiments, the curved portion may be configured to change a direction of the instrument 90° or approximately 90° (or another desired angle) when the instrument 44 is advanced through the connector 75. In some embodiments, the curved portion may include a trough, similar to the trough 68 of FIGS. 2A-2C.

As illustrated, for example, in FIGS. 3A-3D, in some embodiments, the curved portion of the connector 75 may include an enclosed pathway 80. In some embodiments, the enclosed pathway 80 may be disposed within a curved end 82 of the connector 75 having an annular curved outer surface. In some embodiments, when the connector 75 is coupled to the side port 58, the curved end 82 may depress the annular valve 62, which may facilitate advancement of the instrument 44 (see, for example, FIG. 2D) distally through the catheter adapter 50 and the catheter 48 into the vasculature. In some embodiments, when the connector 75 is coupled to the side port 58, the opposing lever arms 79a, 79b may be hooked onto the side port 58, a cap of the side port 58, or another feature. In some embodiments, the opposing lever arms 79a, 79b may be on opposite sides of the longitudinal axis 60, which may facilitate pinching of the opposing lever arms 79a, 79b by a clinician.

As illustrated, for example, in FIG. 3D, in some embodiments, the enclosed pathway 80 may be disposed within a straight end 84 of the connector 75. In some embodiments, the enclosed pathway 80 may exit the connector 75 at a side aperture in the straight end 84. In some embodiments, the connector 75 may include a male luer 86 configured to form a seal within the side port 58, and the straight end 84 may extend from the male luer 86. In some embodiments, the connector 75 may include the male luer 86 configured to form the seal within the side port 58, and the curved end 82 of FIGS. 3A-3D may extend from the male luer 86. In some embodiments, in response to the connector 75 being coupled to the side port 58, the straight end 84 may depress the annular valve 62, which may facilitate advancement of the instrument 44 distally through the catheter adapter 50 and the catheter 48 into the vasculature.

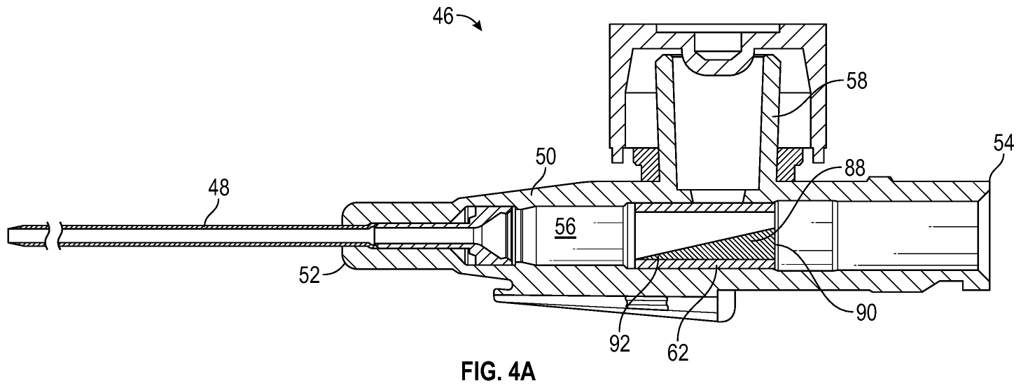

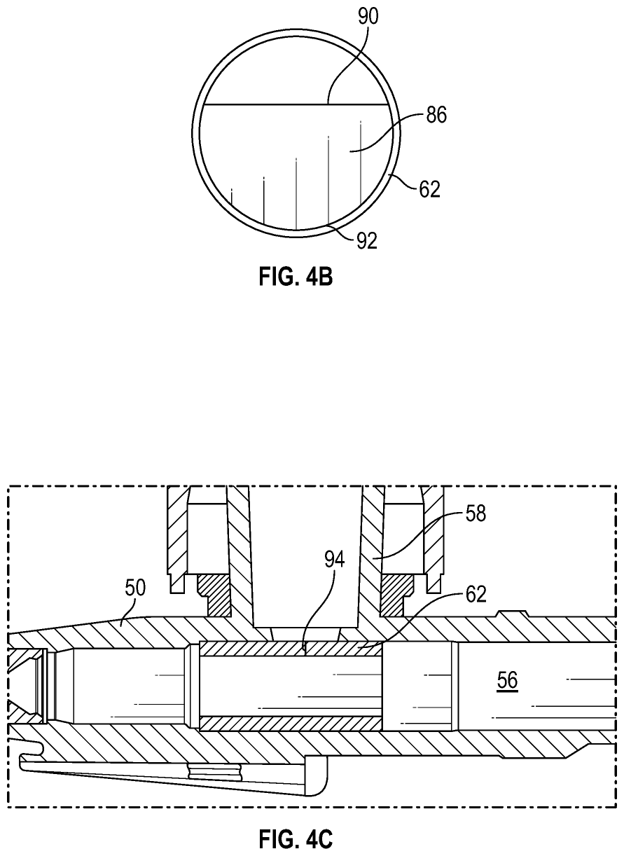

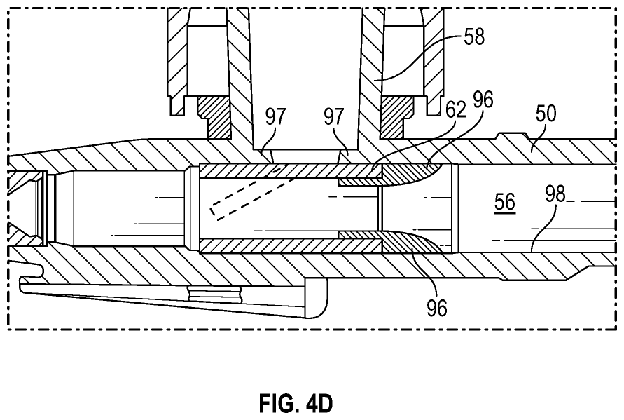

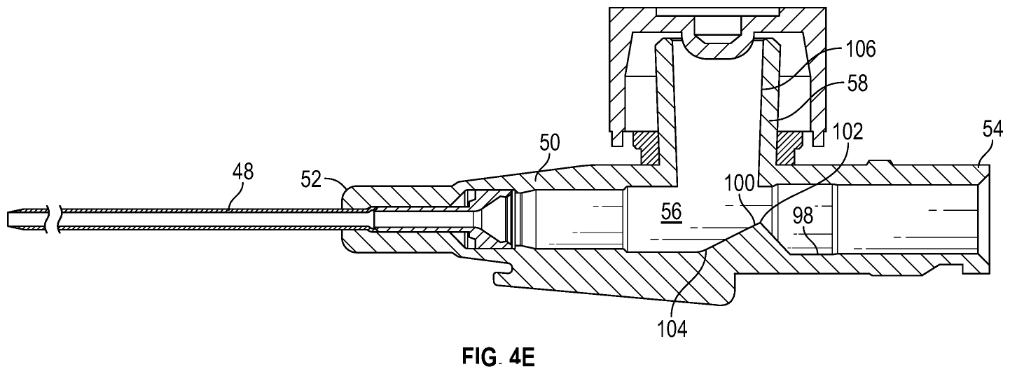

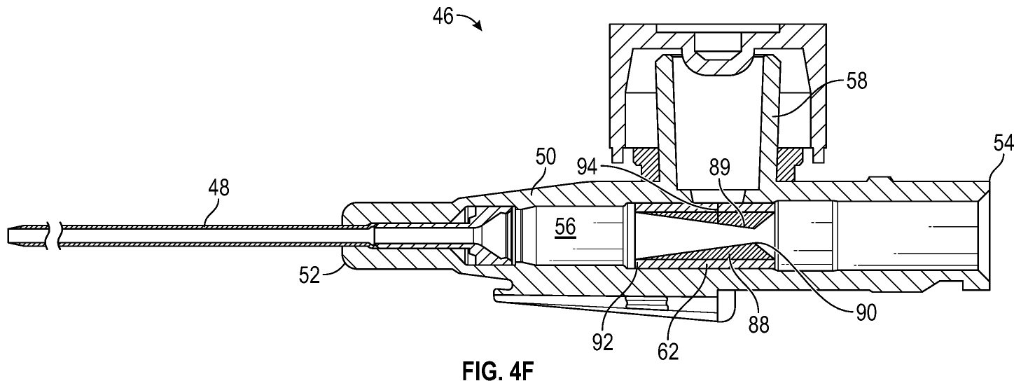

Referring now to FIGS. 4A-4B, in some embodiments, the instrument advancement feature may include a ramp 88 disposed within an interior of the annular valve 62. In some embodiments, a proximal end 90 of the ramp 88 may be higher or thicker than a distal end 92 of the ramp 88 such that a proximal end of the annular valve 62 may depress less than a distal end of the annular valve 62 in response to contact by the instrument 44 (see, for example, FIG. 2D) inserted through the side port 58. Thus, the ramp 88 may facilitate movement of the instrument 44 into the lumen 56 at the distal end of the annular valve 62 and toward a distal end of the catheter system 46. In some embodiments, an upper surface of the ramp 88 may be smooth or planar, which may facilitate movement of the instrument 44 over the annular valve 62 and the ramp 88. In some embodiments, the ramp 88 may be monolithically formed with the annular valve 62 as a single unit.

Referring now to FIG. 4C, in some embodiments, the annular valve 62 may include a slit 94 such that the instrument 44 (see, for example, FIG. 2D) may be inserted into the lumen 56 through the slit 94 and advanced distally. In some embodiments, the slit 94 creates a pathway for the instrument 44 to enter the lumen 56.

Referring now to FIG. 4D, in some embodiments, the instrument advancement feature may include an attachment sleeve 96 that attaches a proximal end of the annular valve 62 to an inner surface 98 of the catheter adapter 50. In some embodiments, the inner surface 98 may form the lumen 56. In some embodiments, the attachment sleeve 96 may be annular. In some embodiments, the attachment sleeve 96 may be coupled to the proximal end of the annular valve 62 and/or the inner surface 98 via an adhesive or another attachment mechanism. In some embodiments, the annular valve 62 and/or the attachment sleeve 96 may be constructed of silicon, plastic, or another suitable material. In some embodiments, the attachment sleeve 96 may cause the annular valve 62 to collapse towards a distal end of the annular valve 62 when the instrument 44 (see, for example, FIG. 2D) contacts the annular valve 62 from the side port 58. In some embodiments, collapsing of the distal end of the annular valve 62 in this manner may form a ramp to guide the instrument 44 distally into the lumen 56 and aid in turning a corner from the side port 58.

In some embodiments, instead of the attachment sleeve 96, an adhesive applied to the proximal end of the annular valve 62 may adhere the proximal end of the annular valve 62 to the inner surface 98 and cause the annular valve 62 to collapse towards a distal end of the annular valve 62 when the instrument contacts the annular valve 62 from the side port 58, ensuring the instrument 44 passes distally through the catheter assembly.

Referring now to FIG. 4E, in some embodiments, the instrument advancement feature may include a ramp 100 on the inner surface 98 of the catheter adapter 50. In some embodiments, a proximal end 102 of the ramp 100 may be higher or thicker than a distal end 104 of the ramp 100. In some embodiments, an upper surface of the ramp 100 may be planar or smooth to facilitate movement of the instrument 44 (see, for example, FIG. 2D) over the ramp 100. In some embodiments, the proximal end 102 of the ramp 100 may be aligned with or proximal to a proximal wall 106 of the side port 58 such that the instrument 44 does not get trapped proximal to the ramp 100. In some embodiments, the ramp 100 may be spaced apart from an opposite wall of the catheter adapter 50, which may facilitate free passage of fluid, tubes, or other devices from the proximal end 54 of the catheter adapter 50.

In some embodiments, one or more protrusions 97 (see, for example, FIG. 4D) extending towards a central axis of the side port 58 may be removed such that the instrument 44 does not get caught on the protrusions 97 as the instrument 44 enters the lumen 56 from the side port 58.

Referring now to FIG. 4F, in some embodiments, the ramp 88 may be disposed on a bottom of the annular valve 62 or a side of the annular valve 62 further from the side port 58. In some embodiments, the ramp 88 may be a first ramp, and a second ramp 89 may be disposed opposite the first ramp within the annular valve 62. In some embodiments, the first ramp may be similar in shape to the second ramp 89 such that the first ramp and the second ramp 89 are symmetric within the annular valve 62. The first ramp and the second ramp 89 may be spaced apart to allow passage of fluid, a tube, or another device therethrough from the proximal end 54 of the catheter adapter 50.

It should be understood that the embodiments may be combined. For example, the annular valve 62 illustrated in FIG. 4F may be coupled to the attachment sleeve 96 of FIG. 4D. In some embodiments, the slit 94 may extend through the annular valve 62 and the second ramp 89. In some embodiments, the first ramp and/or the second ramp 89 may be monolithically formed with the annular valve 62 as a single unit.

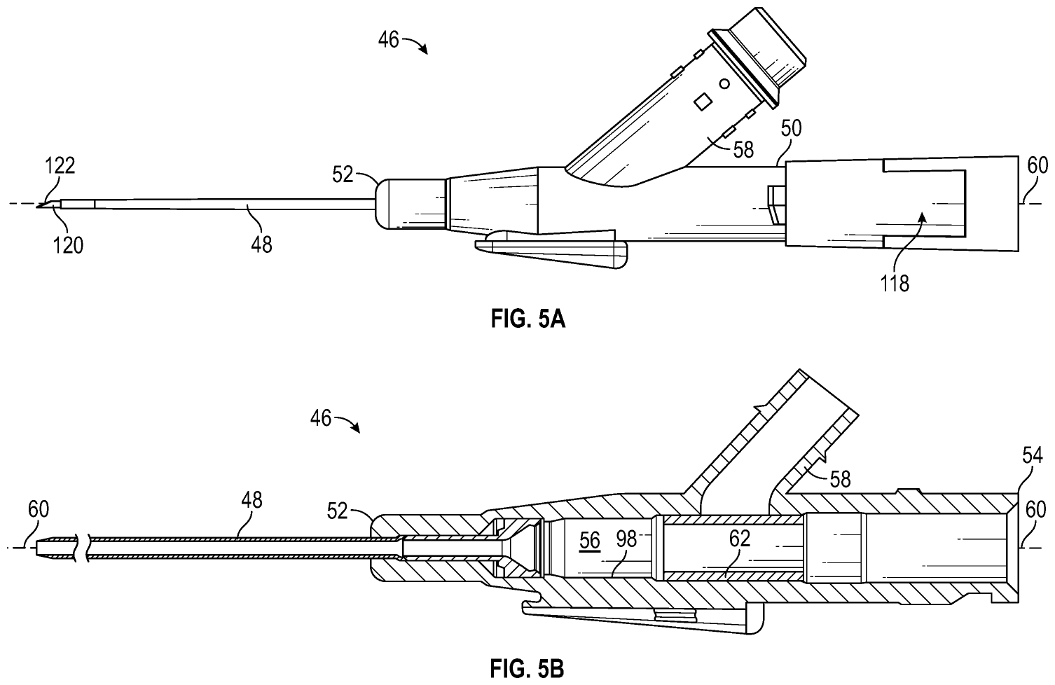

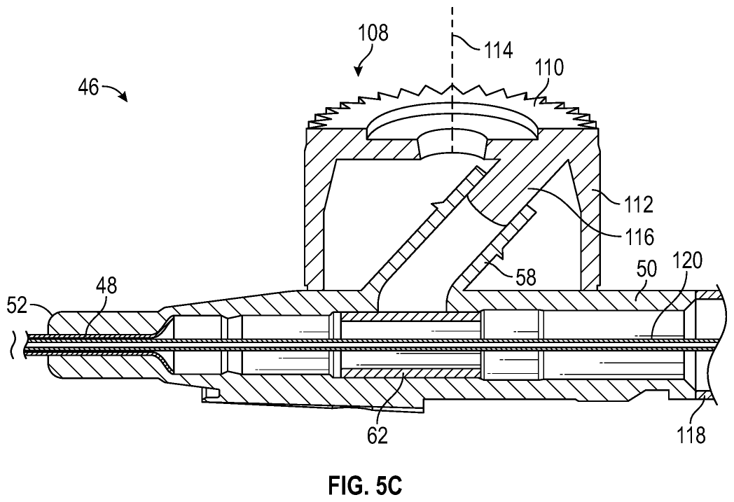

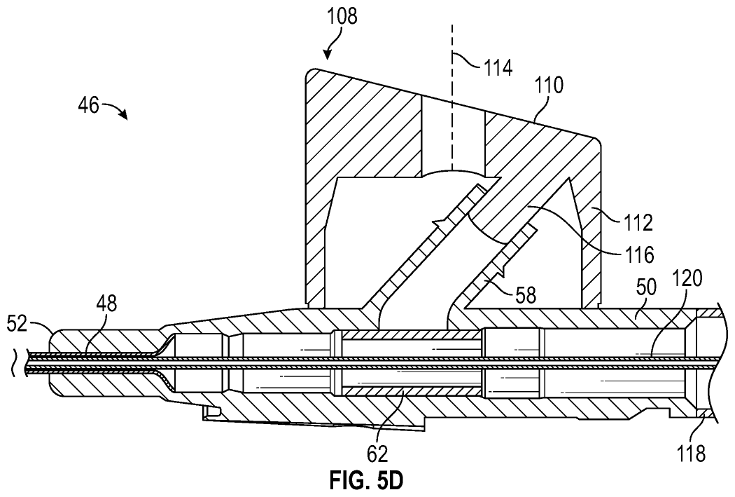

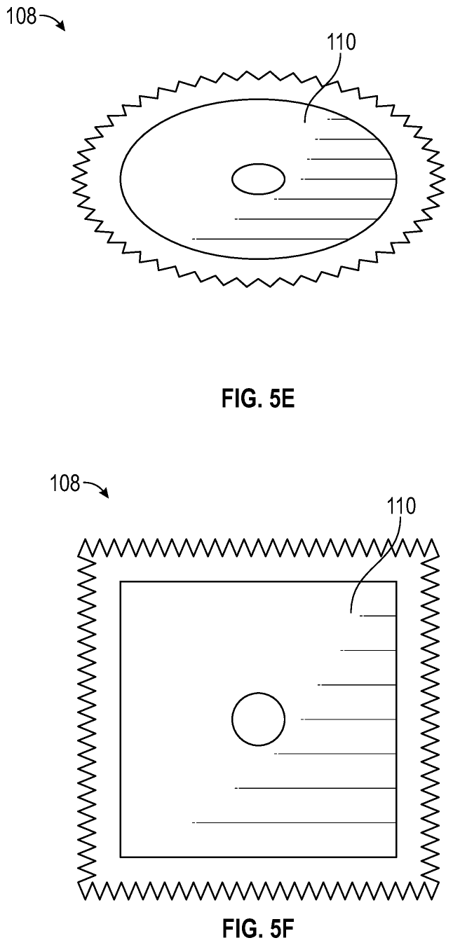

Referring now to FIGS. 5A-5E, in some embodiments, the side port 58 may be fixed with respect to the distal end 52 of the catheter adapter 50 and the proximal end 54 of the catheter adapter 50 at an angle less than 90° with respect to the longitudinal axis 60 of the catheter adapter 50. For example, the side port 58 may be fixed with respect to the distal end 52 of the catheter adapter 50 and the proximal end 54 of the catheter adapter 50 at 60°, 45°, 50°, 25°, or another suitable angle with respect to the longitudinal axis 60 of the catheter adapter 50. In further detail, a central axis of the side port 58 may be fixed with respect to the distal end 52 of the catheter adapter 50 and the proximal end 54 of the catheter adapter 50 at 60°, 45°, 50°, 25°, or another suitable angle with respect to the longitudinal axis 60 of the catheter adapter 50. In some embodiments, the catheter adapter 50 may be monolithically formed as a single unit and/or constructed of a rigid material, such as rigid plastic.

In some embodiments, the catheter system 46 may include a cap 108 coupled to the side port 58. In some embodiments, the cap 108 may include a base 110 and an annular collar 112 extending from the base 110. In some embodiments, a central axis 114 of the cap 108 may be perpendicular to the longitudinal axis 60 of the catheter adapter 50. In some embodiments, an inner surface of the base 110 of the cap 108 may include a protrusion 116 sealing the side port 58. In some embodiments, because an outer surface of the cap 108 is straight or symmetric about the central axis 114 perpendicular to the longitudinal axis 60, the cap 108 may facilitate gripping during insertion of the catheter 48 into the patient. In some embodiments, an inside of the cap 108 may be asymmetric due to the protrusion 116.

In some embodiments, the side port 58 may be located on a top of the catheter adapter 50 or a side furthest away from skin of the patient when the catheter system 46 is inserted into the vasculature of the patient. In some embodiments, the side port 58 disposed on top of the catheter adapter 50 may provide easy access for delivery of the instrument 44 (see, for example, FIG. 2D) through the catheter adapter 50 into the blood vessel. In some embodiments, the catheter system 46 may include a needle hub 118 and an introducer needle 120 extending distally from the needle hub 118. In some embodiments, the introducer needle 120 may include a sharp distal tip configured to pierce skin and vasculature of the patient to insert the catheter 48 through the skin into the vasculature. In some embodiments, the introducer needle 120 may include a bevel 122 forming the sharp distal tip, and the bevel 122 may be facing upward or towards a top of the catheter system 46 in an insertion position ready for insertion into a patient, as illustrated, for example, in FIG. 5A.

As illustrated, for example, in FIG. 5D, in some embodiments, the base 110 of the cap 108 may be ramped or inclined in a distal direction. In further detail, an inclined upper surface of the base 110 may provide a push feature on which the clinician may push distally when inserting the catheter system 46 into the patient. As illustrated, for example, in FIGS. 5E-5F, in some embodiments, a perimeter of the cap 108 may be non-circular, such as oval, square, or rectangular, which may aid the cap 108 is covering the side port 58 disposed at an angle. In these embodiments, the perimeter or shape of the cap 108 may facilitate gripping during insertion of the catheter 48 into the patient while still allowing coverage of the side port 58 by the cap 108.

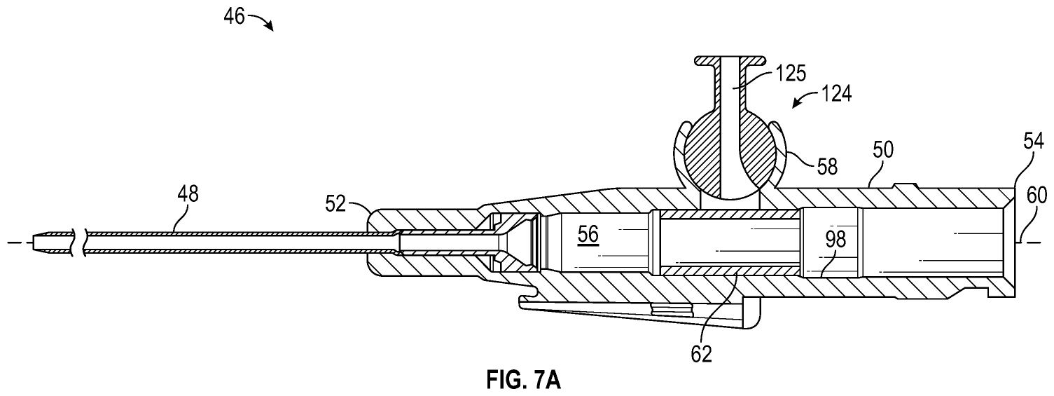

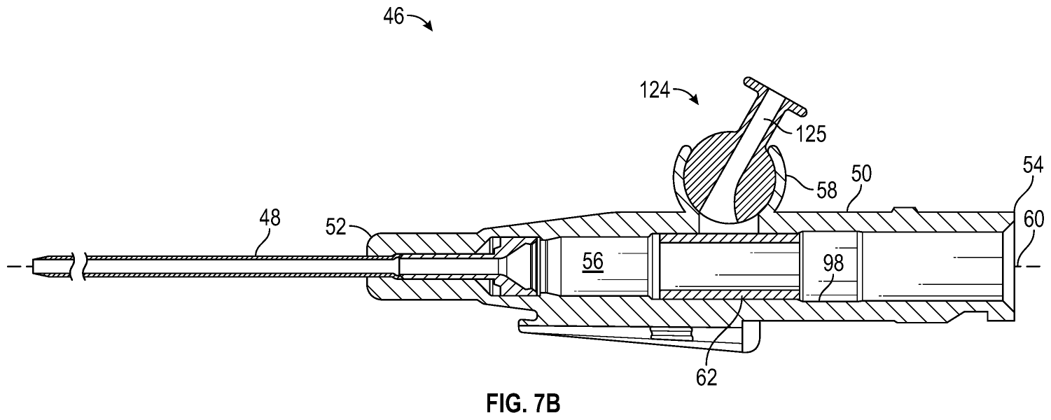

In some embodiments, the side port 58 include a movable joint (see, for example FIGS. 7A-7B), such as a hinge, a ball and socket, a cylinder and socket, which may facilitate movement of the side port 58 from a straight position perpendicular to the longitudinal axis 60 to an angled position with respect to the longitudinal axis 60. In these and other embodiments, an outer surface of the catheter adapter 50 distal to the side port 58 may include a push tab, which may be gripped to facilitate insertion of the catheter 48 into the patient.

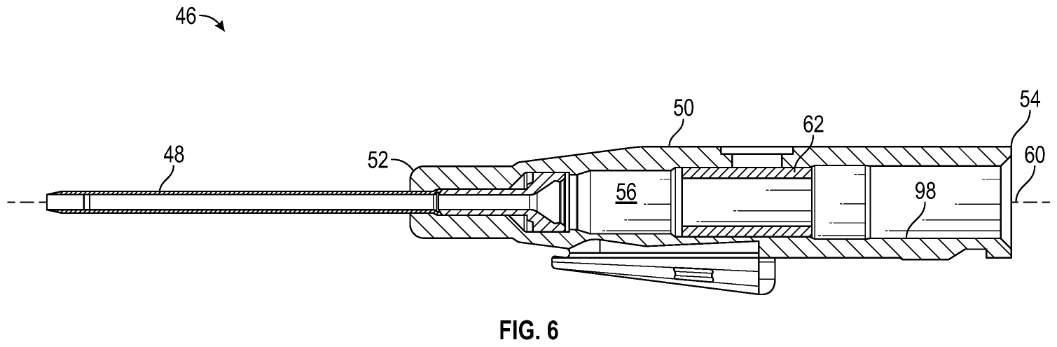

Referring now to FIG. 6, in some embodiments, the catheter adapter 50 may not include the side port 58, which may provide direct access to the lumen 56 by the instrument 44 (see, for example, FIG. 2D). In these embodiments, a top of the catheter adapter 50 may include a planar and/or smooth surface, which may include an aperture therethrough. In some embodiments, the aperture may be aligned with and proximate the annular valve 62.

Referring now to FIGS. 7A-7B, in some embodiments, the side port 58 may include a movable joint 124, which may be movable between a straight position (illustrated, for example, in FIG. 7A) and an angled position (illustrated, for example, in FIG. 7B). In some embodiments, the movable joint 124 may be locked in the straight position and/or the angled position to prevent movement of the movable joint 124 during insertion of the catheter 48 into the patient, for example. In some embodiments, the movable joint 124 may be moved to the angled position to facilitate delivery of the instrument 44 through the side port 58 and distally through the catheter 48. In some embodiments, when the movable joint 124 is in the angled position, the side port 58 may be pointed proximally and angled at less than 90° with respect to the longitudinal axis 60 of the catheter adapter 50.

In some embodiments, the movable joint 124 may include a ball and socket or a cylinder and socket. In further detail, in some embodiments, the movable joint 124 may include a ball portion, which may include a generally spherical outer surface. In some embodiments, the ball portion may be configured to rotate within a socket, which may include a generally spherical inner surface. In some embodiments, the ball portion and the socket may fit together snugly to prevent fluid leakage through the movable joint 124.

In some embodiments, a portion of the side port lumen 125 extending through the ball portion may be shaped such that the instrument 44 does not get caught or trapped as the instrument 44 is inserted through the side port 58. For example, when the movable joint 124 is in an angled position, as illustrated in FIG. 7B, a distal edge of the portion of the side port lumen 125 may be aligned with or proximal to a distal end of an aperture through the catheter adapter 50 such a ledge is not formed on which the instrument 44 may otherwise get caught.

In some embodiments, the movable joint 124 may include a cylinder portion as opposed to the ball portion. In some embodiments, the cylinder portion may be configured to rotate within the socket, which may be generally cylindrical. For further detail on an example ball and socket or cylinder and socket see U.S. Pat. No. 11,590,323, filed May 4, 2020, entitled “CATHETER ASSEMBLY SUPPORT DEVICE, SYSTEMS, AND METHODS,” which is hereby incorporated by reference in its entirety.

In some embodiments, the side port 58 may be constructed of a thinner and/or more flexible material than a remaining portion of the catheter adapter 50, which would allow the side port 58 to flex from a first angle to a second angle with respect to the longitudinal axis 60 of the catheter adapter 50.

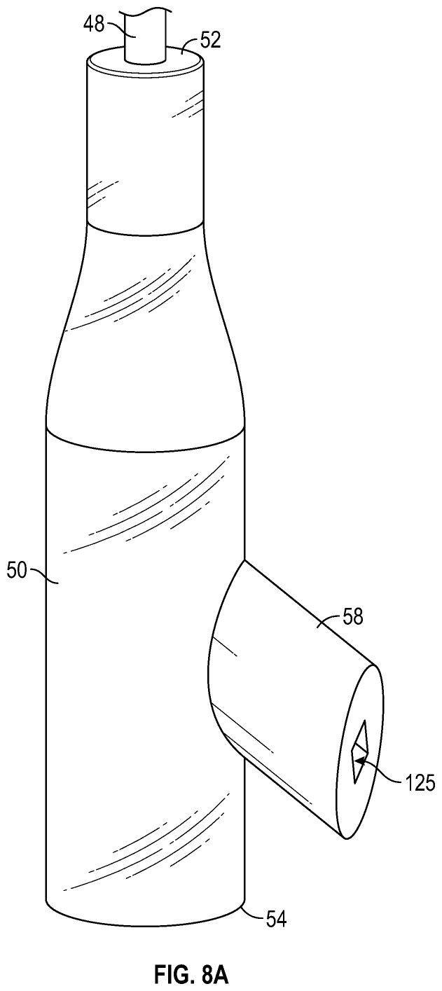

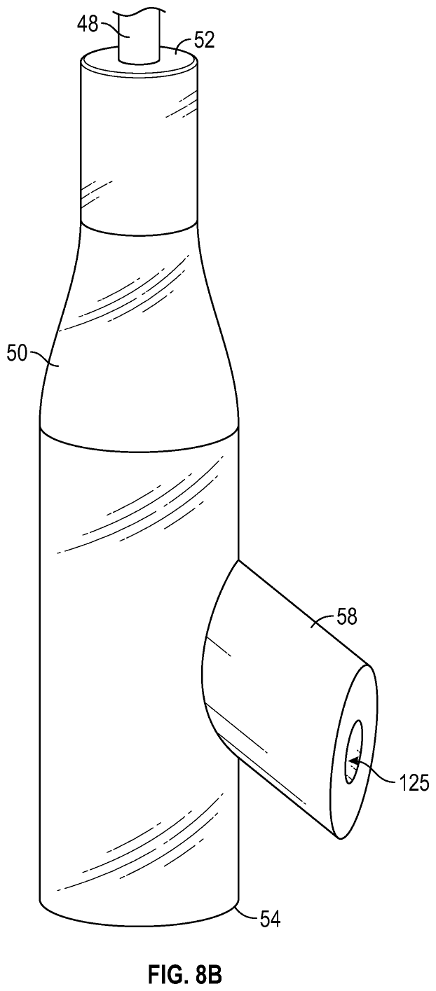

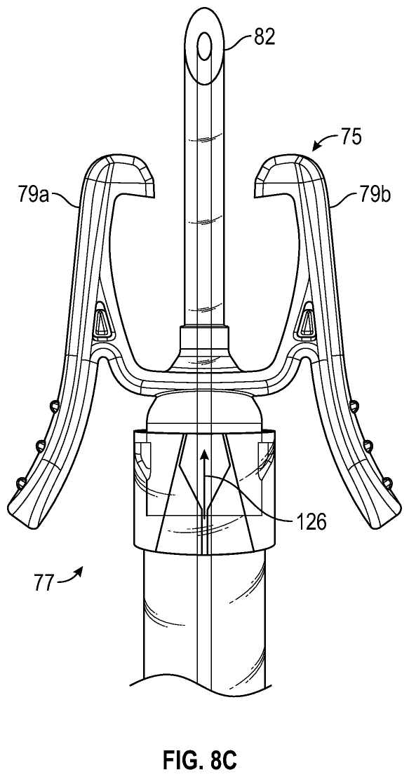

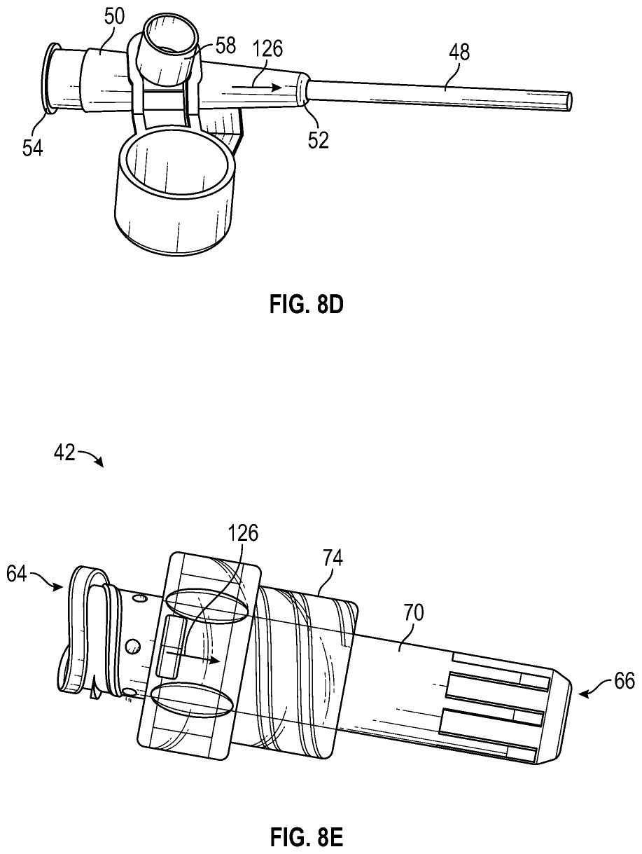

Referring now to FIGS. 8A-8B, in some embodiments, the side port 58 may include a side port lumen 125 having an asymmetric shape. In some embodiments, all or a portion of the side port lumen 125 may include the asymmetric shape. In these embodiments, the side port lumen 125 may facilitate positioning of the connector 75 of the instrument delivery device 77 (see, for example, FIGS. 3A-3C) in a desired orientation such that the instrument advancement feature directs the instrument 44 distally within the lumen of the catheter adapter 50. The asymmetric shape may thus facilitate a “lock and key” fit between the instrument delivery device 77 and the side port 58. In some embodiments, the asymmetric shape may include an oval, a triangle, a diamond, or another suitable shape.

Referring now to FIG. 8C, in some embodiments, the instrument delivery device 77 may include a directional indicator 126 to indicate to the clinician an orientation of the instrument delivery device 77 with respect to the catheter assembly such that the curved portion or other instrument advancement feature is configured to direct the instrument in a distal direction within the catheter assembly. In some embodiments, the directional indicator 126 may include an arrow or another suitable indicator. In some embodiments, the arrow or other suitable indicator may point towards the catheter 48 or catheter assembly. In some embodiments, a particular directional indicator 126 on the side port 58 or catheter assembly may be configured to match or align with another particular directional indicator 126 on the instrument delivery device 77 to indicate a proper orientation of the instrument delivery device 77 with respect to the side port 58 or catheter assembly. In some embodiments, the particular direction indicator 126 may include a first color and the another particular directional indicator 126 may include a second color, which may be a different shade of the first color, for example.

Referring now to FIG. 8D, in some embodiments, the catheter adapter 50 may include the directional indicator 126 to indicate to the clinician an orientation of the instrument delivery device 77 with respect to the catheter assembly such that the curved portion or other instrument advancement feature is configured to direct the instrument in a distal direction within the catheter assembly.

Referring now to FIG. 8E, in some embodiments, the needleless access connector 42 may include the directional indicator 126 to indicate to the clinician an orientation of the instrument delivery device 77 with respect to the catheter assembly such that the curved portion or other instrument advancement feature is configured to direct the instrument in a distal direction within the catheter assembly.

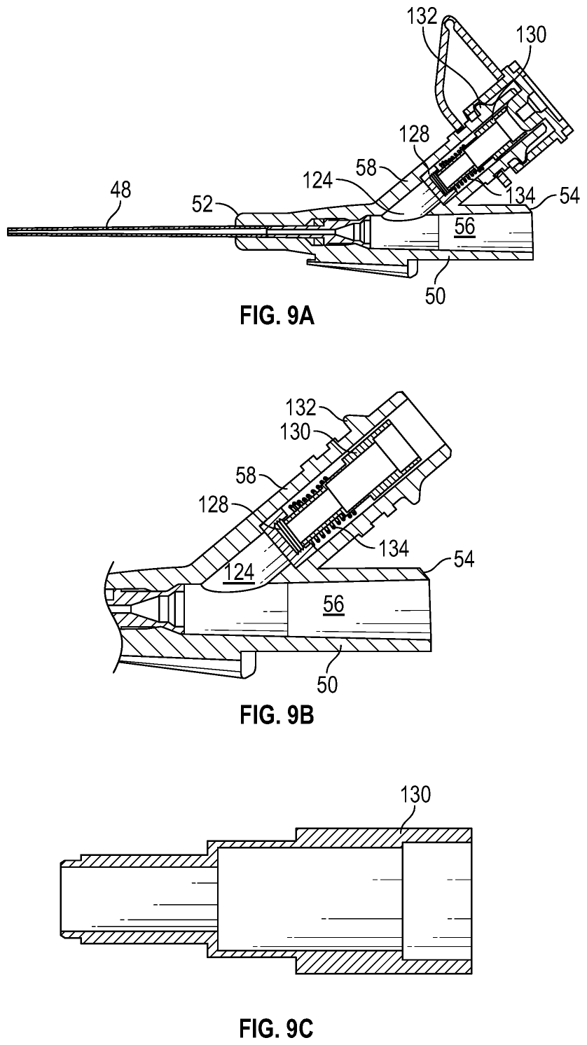

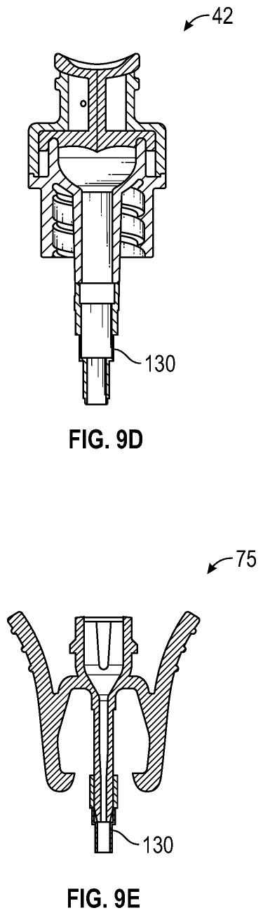

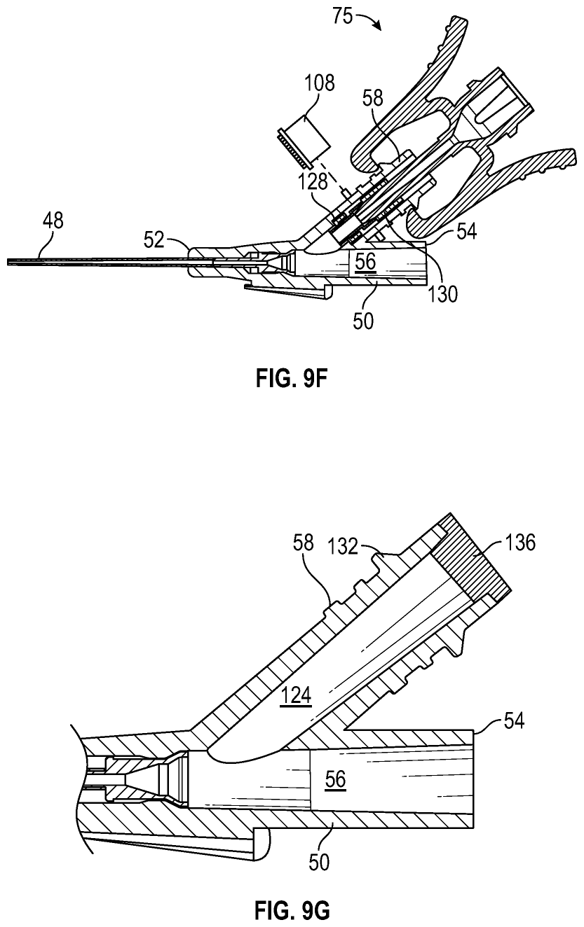

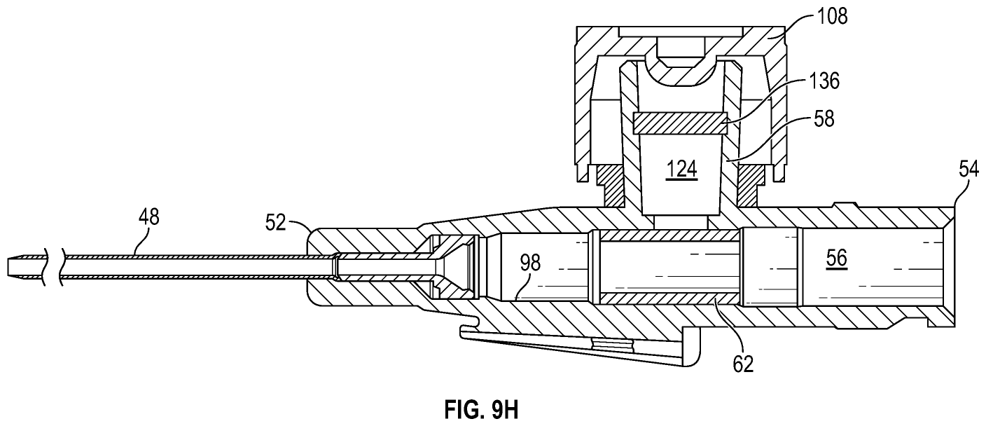

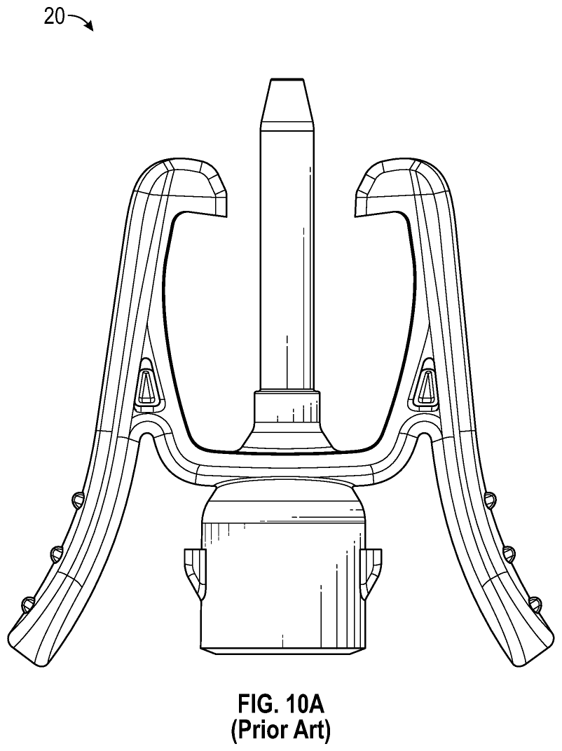

Referring now to FIGS. 9A-9C, in some embodiments, a split septum 128 and/or a septum actuator 130 may be disposed within the side port 58. In these and other embodiments, the annular valve 62 may not be present. In some embodiments, a spring 134 may be coupled to the septum actuator 130 and configured to return the septum actuator 130 to a first position from a second position after a medical device is removed from the side port 58. Thus, the septum actuator 130 may be used multiple times. In some embodiments, the septum actuator 130 may be advanced through the split septum 128 to open the split septum 128 in response to coupling of the medical device to the side port 58. In some embodiments, the medical device may include the instrument delivery device 77 or another suitable medical device. In some embodiments, the split septum 128 may avoid backflow of blood or a drug when the side port 58 is not in use.

Referring now to FIG. 9D-9F, in some embodiments, the septum actuator 130 may be coupled to the needleless access connector 42 or the connector 75. In some embodiments, the septum actuator 130 may be coupled to the needleless access connector 42 or the connector 75 via an adhesive, an interference fit, or another suitable method. In these embodiments, the septum actuator 130 may be inserted into the side port 58 in response to coupling of the needleless access connector 42 or the connector 75 to the side port 58. In some embodiments, as illustrated, for example, in FIG. 9F, the septum actuator 130 may be inserted through the split septum 128 to open the split septum 128 in response to coupling of the needleless access connector 42 or the connector 75 to the side port 58.

In some embodiments, the septum actuator 130 may include an internal step, and an end of a proboscis or insertion portion of the connector 75 may abut the internal step. In some embodiments, coupling of the septum actuator 130 to the needleless access connector 42 or the connector 75 may reduce a need for venous compression during insertion and blood draws, reducing a risk of blood exposure.

Referring now to FIG. 9G, in some embodiments, a septum 136 may be disposed at an end of the side port 58. In some embodiments, the septum 136 may include a split septum having a slit therethrough. In some embodiments, the septum 136 may cover the end of the side port 58 and/or be disposed within the side port lumen 125 in an interference fit. In some embodiments, the side port 58 may include an annular protrusion configured to receive opposing lever arms of a connector. Referring now to FIG. 9H, in some embodiments, the septum 136 may be disposed within the side port lumen 125 and/or spaced apart from the end of the side port 58. In some embodiments, the septum 136 may prevent microbial ingress.

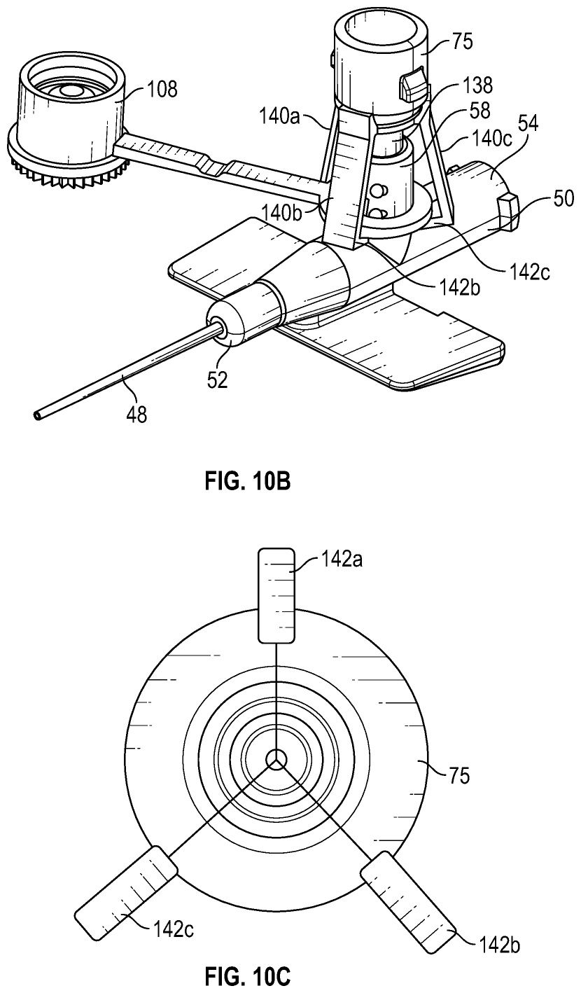

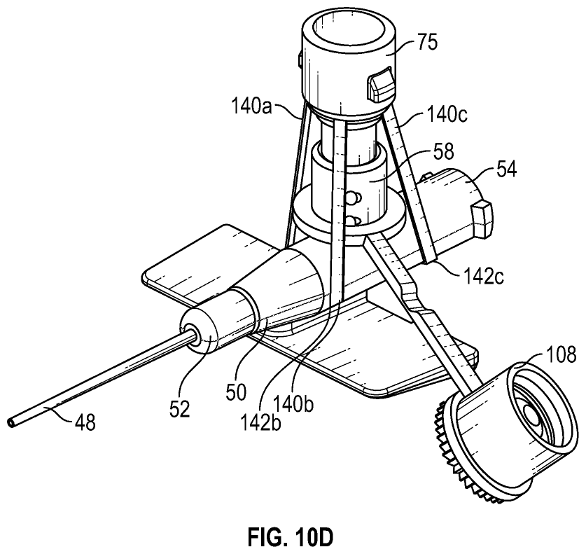

FIG. 10A illustrates the connector 20 of the prior art blood draw device 10. Referring now to FIGS. 10B-10D, in some embodiments, the connector 75 may include the proboscis or insertion portion 138 and multiple arms 140. In some embodiments, the insertion portion 138 may be configured to insert into the catheter adapter 50 and/or form a seal with the catheter adapter 50. In some embodiments, the multiple arms 140 may be configured to couple to or snap onto an exterior of the catheter assembly, such as the cap 108 (see FIG. 10B, for example) or the catheter adapter 50 (see FIG. 10D, for example). In some embodiments, the multiple arms 140 may be snapped onto an annular connector of the cap 108 that couples the cap 108 to the side port 58. In some embodiments, an end of each of the multiple arms 140 may include a flange 142 to facilitate the snapping onto the cap 108 or the catheter adapter 50.

In some embodiments, the multiple arms 140 may include three arms. In some embodiments, the multiple arms 140 may include more than three arms. In some embodiments, a tether of the cap 108 may extend between the annular connector and a cover portion of the cap 108 configured to cover and/or seal an opening. In some embodiments, the tether may extend between two arms of the multiple arms 140, which may be coupled to a same side of the catheter adapter 50. In these and other embodiments, a single arm of the multiple arms 140 may be coupled to an opposite side of the catheter adapter 50 than the two arms. In some embodiments, the single arm may be positioned in between the two arms to provide stability to the connection between the connector 20 and the catheter adapter 50.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. An instrument delivery device to insert an instrument through a catheter assembly, the instrument delivery device comprising:
a housing, comprising a proximal end, a distal end, and a slot disposed between the proximal end of the housing and the distal end of the housing;
a connector coupled to the distal end of the housing;
a slider configured to move along the slot; and
an instrument disposed within the housing and coupled to the slider, wherein the instrument is configured to advance beyond the connector in response to movement of the slider along the slot from a proximal position to a distal position,
wherein the connector comprises a curved portion configured to change a direction of the instrument when the instrument is advanced through the connector.

2. The instrument delivery device of claim 1, wherein the curved portion is configured to change a direction of the instrument 90° when the instrument is advanced through the connector.

3. The instrument delivery device of claim 1, wherein the curved portion comprises a trough.

4. The instrument delivery device of claim 1, wherein the curved portion comprises an enclosed pathway.

5. The instrument delivery device of claim 4, wherein the enclosed pathway is disposed within a straight end of the connector.

6. The instrument delivery device of claim 5, wherein the connector comprises a male luer configured to form a seal within the catheter assembly, wherein the straight end extends from the male luer.

7. The instrument delivery device of claim 4, wherein the enclosed pathway is disposed within a curved end of the connector.

8. The instrument delivery device of claim 7, wherein the connector comprises a male luer configured to form a seal within the catheter assembly, wherein the curved end extends from the male luer.

9. The instrument delivery device of claim 1, further comprising a directional indicator to indicate to a clinician an orientation of the instrument delivery device with respect to the catheter assembly such that the curved portion is configured to direct the instrument in a distal direction within the catheter assembly.

10. The instrument delivery device of claim 1, wherein the connector comprises an insertion portion and a plurality of arms, wherein the insertion portion is configured to insert into the catheter assembly, wherein the plurality of arms are configured to snap onto an exterior of the catheter assembly.

11. A catheter system, comprising:
a catheter adapter, comprising a distal end, a proximal end, a lumen extending through the distal end of the catheter adapter and the proximal end of the catheter adapter, and a side port disposed between the distal end of the catheter adapter and the proximal end of the catheter adapter;
a catheter extending from the distal end of the catheter adapter;
an annular valve disposed within the lumen and configured to seal the side port; and
an instrument advancement feature, wherein when an instrument is inserted through the side port, the instrument advancement feature is configured to direct the instrument in a distal direction within the lumen of the catheter adapter.

12. The catheter system of claim 11, wherein the instrument advancement feature comprises a ramp disposed within an interior of the annular valve.

13. The catheter system of claim 11, wherein the instrument advancement feature comprises an attachment sleeve or adhesive that attaches a proximal end of the annular valve to an inner surface of the catheter adapter.

14. The catheter system of claim 11, wherein the instrument advancement feature comprises a ramp on an inner surface of the catheter adapter.

15. The catheter system of claim 11, further comprising a needleless access connector, comprising:
a first end comprising a female luer; and
a second end coupled to the side port, wherein the second end comprises the instrument advancement feature, wherein the instrument advancement feature comprises a curved portion, wherein the curved portion comprises a trough or an enclosed pathway.

16. A catheter system, comprising:
a catheter adapter, comprising a distal end, a proximal end, a lumen extending through the distal end of the catheter adapter and the proximal end of the catheter adapter, and a side port disposed between the distal end and the proximal end, wherein the side port is configured to be angled at less than 90° with respect to a longitudinal axis of the catheter adapter;
a catheter extending from the distal end of the catheter adapter; and
an annular valve disposed within the lumen and configured to seal the side port.

17. The catheter system of claim 16, wherein the side port is fixed with respect to the distal end of the catheter adapter and the proximal end of the catheter at an angle less than 90° with respect to the longitudinal axis of the catheter adapter, further comprising a cap coupled to the side port, wherein a central axis of the cap is perpendicular to the longitudinal axis of the catheter adapter, wherein an inner surface of the cap comprises a protrusion sealing the side port.

18. The catheter system of claim 16, wherein the side port comprises a movable joint.

19. The catheter system of claim 16, wherein the side port comprises a side port lumen having an asymmetric shape.

20. The catheter system of claim 16, wherein the side port is fixed with respect to the distal end of the catheter adapter and the proximal end of the catheter adapter at an angle less than 90° with respect to the longitudinal axis of the catheter adapter, wherein the side port extends from a top of catheter adapter, further comprising a septum disposed within the side port.
